(19) United States
(12) Patent Application Publication
(10) Pub. No.: US 2025/0017484 A1
(43) Pub. Date: Jan. 16, 2025

(54) BIOIMPEDANCE MEASUREMENT DEVICE AND METHOD

(71) Applicants: Hologic, Inc., Marlborough, MA (US); University of Hawaii, Honolulu, HI (US)

(72) Inventors: Thomas L. KELLY, Woburn, MA (US); John A. SHEPHERD, Honolulu, HI (US)

(73) Assignees: Hologic, Inc., Marlborough, MA (US); University of Hawaii, Honolulu, HI (US)

(21) Appl. No.: 18/711,190

(22) PCT Filed: Nov. 21, 2022
(86) PCT No.: PCT/US2022/080256

§ 371 Date: May 17, 2024

Related U.S. Application Data
(60) Provisional application No. 63/282,960, filed on Nov. 24, 2021.

(51) Int. Cl. A61B 5/0537; A61B 5/00; A61B 5/01

(52) U.S. Cl. CPC A61B 5/0537; A61B 5/01; A61B 5/443; A61B 5/6801; A61B 5/7271; A61B 2560/0468; A61B 2562/0271

(57) ABSTRACT

In some embodiments, a bioimpedance measurement apparatus includes a set of electrodes configured to be placed on respective skin portions of a subject; a temperature sensor probe configured to be placed on a respective skin portion of the subject; a moisture sensor probe configured to be placed on a respective skin portion of the subject; and an electronic module configured to determine an impedance between at least two of the plurality of electrodes, receive from the temperature probe a temperature signal and determine from the temperature signal a temperature of the skin portion the temperature sensor probe is placed on, receive from the moisture sensor probe a moisture signal and determine a moisture level of the skin portion the moisture sensor probe is placed on, and determine a bioimpedance of the subject based at least in part on the determined impedance, temperature, and moisture level.

### CROSS-REFERENCE TO RELATED APPLICATION

This application is being filed on Nov. 21, 2022, as a PCT International Patent Application that claims priority to and the benefit of U.S. Provisional Patent Application Ser. No. 63/282,960, filed Nov. 24, 2021, which is incorporated by reference in its entirety into the present application.

### BACKGROUND

Bioimpedance devices measure intracellular and extracellular water in vivo by passing small electrical currents through the tissues of subjects and measuring voltage drops along the current paths. Bioimpedance measurements, by itself or injunction with other diagnostic techniques, can be used to ascertain physiological states of subject by providing measurements of water content in the subject. Accurate bioimpedance measurements are thus valuable for medical diagnoses and physical condition assessments.

### SUMMARY

In some embodiments, a bioimpedance measurement apparatus includes a set of electrodes configured to be placed on respective skin portions of a subject; at least one temperature sensor probe configured to be placed on a respective skin portion of the subject and to generate a signal indicative of a temperature of the skin portion the at least one temperature sensor probe is placed on; at least one moisture sensor probe configured to be placed on a respective skin portion of the subject and to generate a signal indicative of a moisture level of the skin portion the at least one moisture sensor probe is placed on; and an electronic module configured to determine an impedance between at least two of the plurality of electrodes, receive from the at least one temperature probe the signal indicative of the temperature of the skin portion the at least one temperature sensor probe is placed on and determine the at least one temperature of the skin portion the temperature sensor probe is placed on, receive from the at least one moisture sensor probe the signal indicative of the moisture level of the skin portion the at least one moisture sensor probe is placed on and determine the moisture level of the skin portion the at least one moisture sensor probe is placed on, and determine a bioimpedance of the subject based at least in part on the determined impedance, temperature, and moisture level.

In some embodiments, a system for determine muscle mass of a subject includes: a radiographic imaging system configured to acquire a radiographic image of the subject; a bioimpedance measurement apparatus, which includes: a set of electrodes configured to be placed on respective skin portions of a subject; at least one temperature sensor probe configured to be placed on a respective skin portion of the subject and to generate a signal indicative of a temperature of the skin portion the at least one temperature sensor probe is placed on; at least one moisture sensor probe configured to be placed on a respective skin portion of the subject and to generate a signal indicative of a moisture level of the skin portion the at least one moisture sensor probe is placed on; and an electronic module configured to: determine an impedance between at least two of the plurality of electrodes, receive from the at least one temperature probe the signal indicative of the temperature of the skin portion the at least one temperature sensor probe is placed on and determine the at least one temperature of the skin portion the temperature sensor probe is placed on, receive from the at least one moisture sensor probe the signal indicative of the moisture level of the skin portion the at least one moisture sensor probe is placed on and determine the moisture level of the skin portion the at least one moisture sensor probe is placed on, and determine a bioimpedance of the subject based at least in part on the determined impedance, temperature, and moisture level. The system further includes a processor configured to: receive from the radiographic imaging system image data representing the acquired image; receive from the electronic module the determined bioimpedance; and determine a muscle mass of the subject based at least in part on the image data and the determined bioimpedance.

In some embodiments, a method of bioimpedance measurement includes: measuring an impedance between two skin portions of a subject; measuring a skin temperature of the subject; measuring a skin moisture level of the subject; and determining a bioimpedance of the subject based at least in part on the measured impedance, temperature, and moisture level.

### BRIEF DESCRIPTION OF THE DRAWINGS

FIG. 1A shows schematically a bioimpedance measurement setting according to some embodiments.

FIG. 1B shows schematically a system for measuring bioimpedance according to some embodiments.

FIG. 2 shows schematically another system for measuring bioimpedance according to some embodiments.

FIG. 3 shows schematically a support for attaching a skin moisture sensor, a skin temperature sensor, and an bioimpedance measurement electrode according to some embodiments.

FIG. 4 shows schematically another support for attaching a skin moisture sensor, a skin temperature sensor, and a bioimpedance measurement electrode according to some embodiments.

FIG. 5 outlines a method of measuring bioimpedance according to some embodiments.

### DETAILED DESCRIPTION

This disclosure relates to bioimpedance measurements. Bioimpedance measurement devices measure intracellular and extracellular water in vivo by passing small electrical currents through the tissues of subjects and measuring voltage drops along the current paths. Bioimpedance measurements can take various forms, including bioimpedance analysis (BIA) and bioimpedance spectroscopy (BIS). Bioimpedance measurements, by itself or in conjunction with other diagnostic techniques, can be used to ascertain physiological states of subject by providing measurements of water content in the subject. For example, certain diagnostic imaging techniques, such as dual-energy X-ray absorptiometry (“DXA”), can be used to distinguish between fat tissue and muscle tissue. However, some of these techniques are insensitive to the differences between water and muscle fibers in the muscle tissues. Water content measurements provided by bioimpedance measurements can be used in conjunction with diagnostic imaging techniques to ascertain true muscle mass, or muscle mass normalized to a specific level of water content. Accurate bioimpedance measurements are thus valuable for medical diagnoses and physical condition assessments.

In certain embodiments, bioimpedance is measure in conjunction with temperature and/or skin moisture measurements in order to more accurately account for the effects of variations in the temperature and skin moisture on bioimpedance measurements. In some embodiments, one or more temperature and/or skin moisture sensors are placed in proximity to one or more respective electrodes for bioimpedance measurement. For example, temperature and/or skin moisture sensors can be placed on the same support (e.g., adhesive pad) as any electrode for bioimpedance measurement. In further examples, an electronic module is connected to the bioimpedance measurement electrodes and temperature and/or skin moisture sensors, and configured to determine the measured bioimpedance from the signals received from the bioimpedance measurement electrode and correct the measured bioimpedance based on the temperature and/or skin moisture measurements from the temperature and/or skin moisture sensors. In further embodiments, the electronic module can be used in conjunction with, or be a part of, a processing unit for a diagnostic imaging apparatus, such as a DXA imaging apparatus, to provide bioimpedance data for determining body composition (e.g., muscle and/or fat masses) corrected (or calibrated) for water content based on the bioimpedance data. In further embodiments, the electronic module can be used in conjunction with, or be a part of, a processing unit for a imaging apparatus, such as a two-dimensional (2D) or three-dimensional (3D) optical imaging apparatus, to provide bioimpedance data for determining body composition (e.g., muscle and/or fat mass) corrected (or calibrated) for water content based on the bioimpedance data.

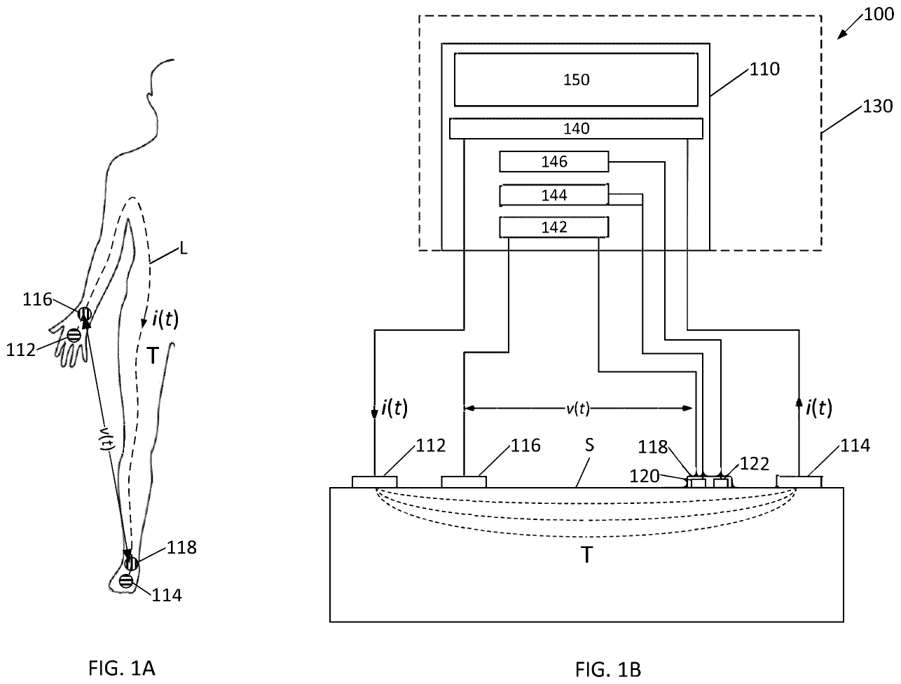

In some embodiments, such as the example shown in FIG. 1A, the impedance of tissues, T, of a subject is measured with a four-probe bioimpedance measurement apparatus, with two current electrodes 112, 114 supplying any electrical current, i(t), which may vary with time, t, through the tissues, T, and to voltage electrodes 116, 118 placed along the current path, L, and between the current electrodes 112, 114. The impedance between the voltage electrodes 116, 118 can be derived from the voltage, v(t), between the two voltage electrodes 116, 118 and the current, i(t). For example, for a DC current, the impedance is simply the resistance between the voltage electros 116, 118, i.e., the ratio between the constant voltage and current; for a sinusoidal AC current, the impedance is the ratio between the amplitudes of the voltage and current, multiplied by a phase factor, which is cosine of the phase angle between the voltage and current.

In some embodiments, such as the example shown FIG. 1B, a system 100 for characterizing at least a portion (T) of a subject includes the bioimpedance measurement electrodes 112, 114, 116, 118 and an electronic module 110 configured to supply a current to the subject through the current electrodes 112, 114, and to measure the voltage between the voltage electrodes 116, 118. The bioimpedance measurement electrodes 112, 114, 116, 118 are adapted to be attached to the subject. For example, a bioimpedance measurement electrode 112, 114, 116, 118 can be electrodes supported by respective pads, which can be adhesive pads attachable to the skin, S, of the subject. Alternatively, the pads can be attached to the skin by other means, such as adhesive tapes or suction cups.

The electronic module 110 in some embodiments includes a current source 140, which supplies a current i(t) through the tissue, T, of the subject through the current electrode's 112, 114. The electron module 110 further includes the voltage sensor 142, which measures the voltage v(t) between the voltage electrodes 116, 118.

The system 100 in FIG. 1B further includes a temperature sensor probe 120. The electronic module 110 in the example further includes a thermometer circuit 144, which receives electrical signals from temperature sensor probe 120 and generates a signal indicative of the temperature at the temperature sensor probe. Any temperature sensor probe suitable for measuring the temperature of the subject and thermometer circuit appropriate for the temperature sensor probe can be used. For example, the temperature sensor probe 120 can be a thermocouple, and the corresponding thermometer circuit 144 can be a voltage sensor, which measures the voltage generated by the thermocouple 120. The thermometer circuit 144 in this case can further includes a temperature reference voltage generator, and measures the voltage generated by the thermocouple 120 against the temperature reference voltage. As another example, the temperature sensor probe 120 can be an infrared (“IR”) sensor, such as an IR photodiode or fiber optic IR temperature sensor, and the corresponding thermometer circuit 144 can be a voltage or current sensor, which measures the voltage or current generated by the IR sensor.

System 100 FIG. 1B further includes a skin moisture sensor probe 122. The electronic module 110 in the example further includes a sensing circuit 146, which receives electrical signals from the skin moisture sensor probe 122 and generates a signal indicative of the skin moisture at the skin moisture sensor probe 122. For example, the skin moisture sensor probe 122 can be a pair of spaced-apart electrodes to be put in contact with the skin, S, of the subject, such that the electrical resistance between the electrodes is indicative of the level of skin moisture at the skin moisture sensor probe 122. The sensing circuit 146 in this example can be a resistance/impedance measurement circuit, which can include a voltage source for applying a voltage across the electrodes in the skin moisture sensor probe 122, and a current sensor to measure the current through the electrodes and the skin portion between the electrodes.

The electronic module 110 in some embodiments further includes a processor 150, which can be a digital processor connected to receive (directly, or indirectly through appropriate intermediate components, such as analog-two-digital converters (“ADC”) and data bus) signals from the voltage sensor 142, thermometer circuit 144, and sensing circuit 146, and programmed to calculate a bioimpedance as sensed using the bioimpedance measurement electrode's 112, 114, 116, 118, and modify the calculated bioimpedance based on the temperature as measured using the temperature probe 120 and/or skin moisture as measured using the skin moisture sensor probe 122. The modification of the measured bioimpedance can be based on previously gained information about the dependence of measured bioimpedance on temperature and/or skin moisture level. Such information can be in the form of analytical formula, which can be incorporated into the program that runs in the processor 150, or one or more lookup tables storing impedance or variations in impedance as a function of temperature and/or skin moisture level. Information about the dependence of measured bioimpedance on temperature and/or skin moisture level can be obtained through a calibration process or from other sources, such as results of studies on the effects of various factors on measured bioimpedance.

In some embodiments, such as in the example shown in FIG. 1B, the temperature sensor probe 120 and the skin moisture sensor probe 122 are disposed in proximity with, or within, one or more bioimpedance measurement electrodes. In the example shown in FIG. 1B, the temperature sensor probe 120 and the skin moisture sensor probe 122 are disposed within the voltage electrode 118. However the sensor probes 120, 122 can be disposed in proximity with, or within, anyone of the bioimpedance measurement electrode's 112, 114, 116, 118. Moreover, temperature and/or skin moisture sensor probes can be disposed in proximity with, or within, more than one bioimpedance measurement electrode. In some embodiments, temperatures and/or skin moisture levels measured at multiple locations can be combined (e.g., averaged) to provide a composite temperature and/or composite skin moisture level. The composite temperature and or skin moisture level can be used to provide the modified bioimpedance.

In some embodiments, the system 100 for characterizing at least a portion (T) of a subject further includes a processor 130 associated with a radiographic imaging apparatus, such as a DXA imaging apparatus. The processor 130 in some embodiments includes a conventional processing unit four a radiographic imaging apparatus and is capable of receiving radiographic imaging data, such as signals from X-ray detectors of a DXA imaging apparatus, and processing the signals to generate radiographic images of a subject, as well as displaying, storing, further processing, and/or transmitting over networks such radiographic images. In some embodiments, the electronic module 110 is integrated into the processor 130. In other embodiments the electronic module 110 is separate from the processor 130. The processor 130 and electronic module 110 is some embodiments share a processor 150; in other embodiments, the processor 130 includes a digital processor separate from the processor 150. In each example disclosed above, they electronic module 110 and processor 130 are connected to each other at least by a data communication the modified bioimpedance measurements produced by the electronic module 110 can be used by the processor 130 to generate information about body composition (e.g., muscle mass) taking into account information provided by the bioimpedance measurements (e.g., water content).

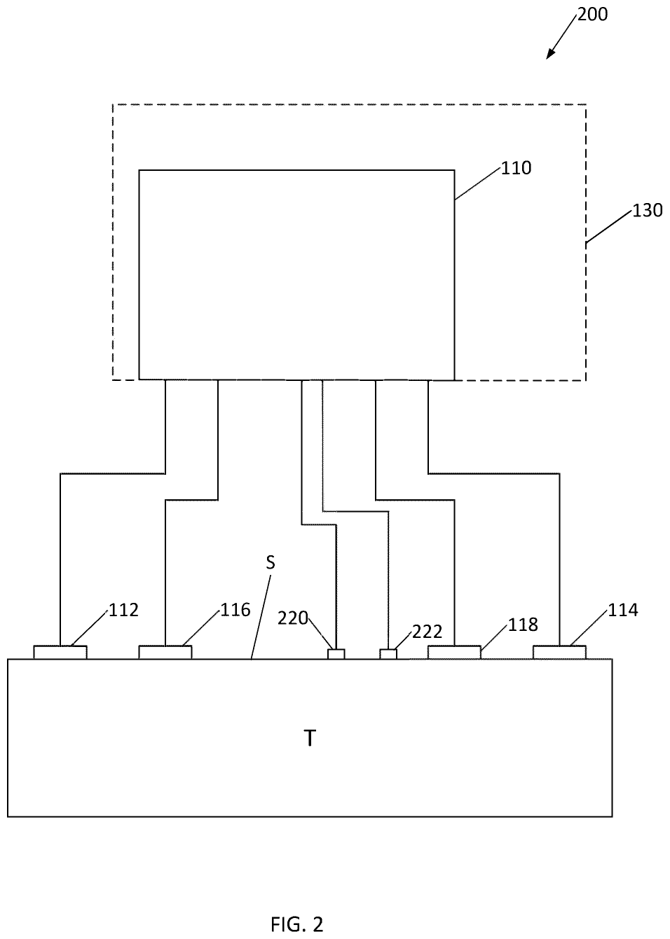

In some embodiments, such as the example shown in FIG. 2, a system 200 for characterizing at least a portion (T) of a subject is otherwise similar to the system 100 shall mean FIG. 1B, except that the temperature sensor probe 220 and/or skin moisture sensor probe 222 are located outside of the bioimpedance measurement electrodes 112, 114, 116, 118. For example, the temperature sensor probe 220 can be disposed in an area on a human body (e.g., four head or oral cavity) where temperature measurements are deemed sufficiently reliable.

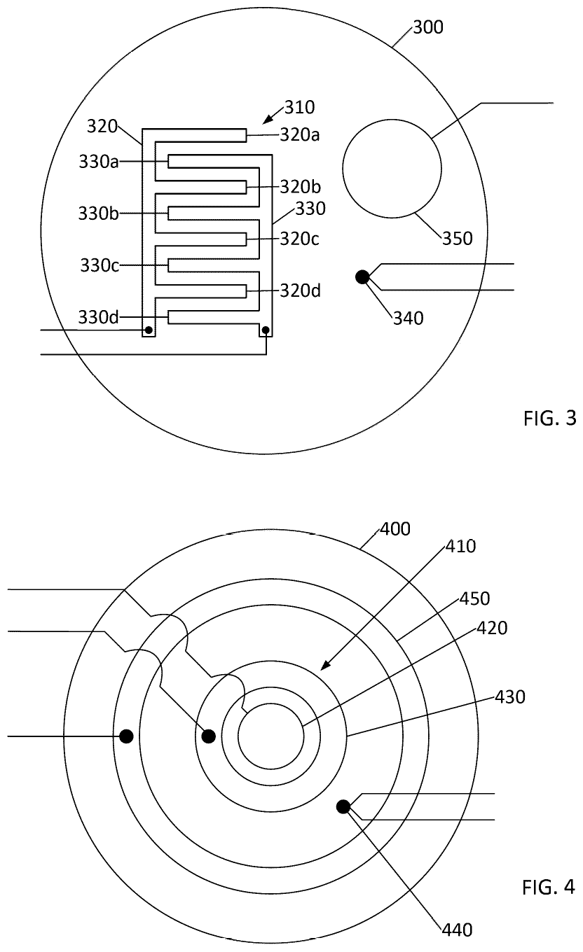

In some embodiments, such as those shown in FIGS. 3 and 4, a bioimpedance measurement electrode, a temperature sensor probe, and/or a skin moisture sensor probe are placed in proximity with each other and place on the same support, such as an adhesive pad for attaching the electrode and probe(s) to the surface (skin) of a subject. As shown in FIG. 3, a skin moisture sensor probe 310, temperature sensor probe's 340, and current or voltage electrode 350 are supported on a shared pad 300. In this example, the skin moisture sensor probe 310 includes a pair of electrodes 320, 330, with interleaved fins 320a-d, 330a-d. The temperature sensor probe 340 in this example includes a thermocouple probe. In another example, shown in FIG. 4, a skin moisture sensor probe 410 and a temperature sensor probe 440 are placed inside a rain-shaped bioimpedance measurement electrode 450; the skin moisture sensor probe 410, temperature sensor probe 440, and bioimpedance measurement electrode 450 are placed on a shared support 400, which can be a pad, such as an adhesive pad for attaching the electrode and probes to the skin of a subject. The skin moisture sensor probe 410 in this example includes a pair of concentric electrodes 420, 430; the temperature sensor probe for 440 in this example includes a thermocouple.

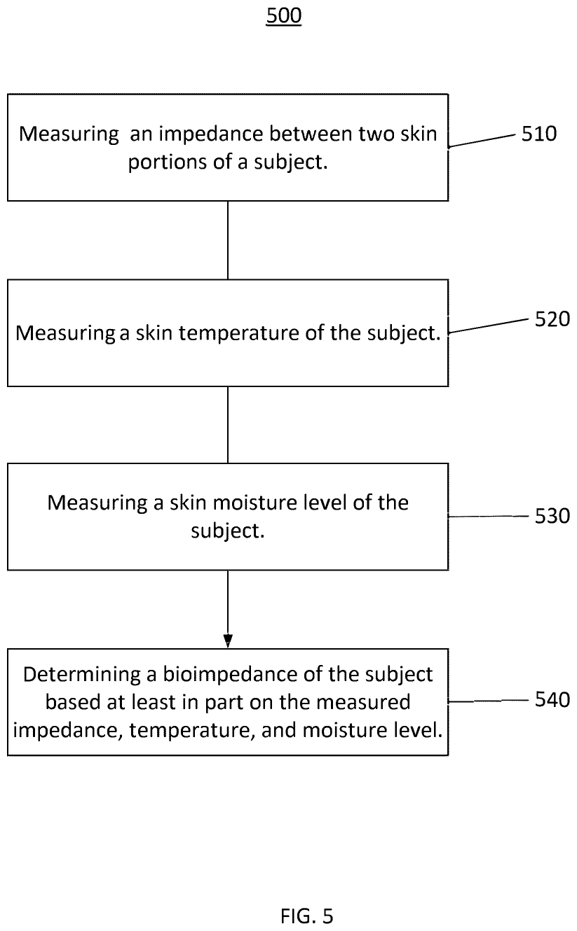

In some embodiments, such as the one outlined in FIG. 5, a method 500 of determining a bioimpedance of at least a portion of a subject includes: measuring 510 an impedance between two skin portions of a subject; measuring 520 a skin temperature of the subject; measuring 530 a skin moisture level of the subject; and determining 540 a bioimpedance of the subject based at least in part on the measured impedance, temperature, and moisture level. In some embodiments, the measuring steps 510, 520, 530 are carried out substantially simultaneously; in other embodiments at least two of the measurement steps 510, 520, 530 are carried out in non-overlapping time periods. Each of the measuring steps 510, 520, 530 can be carried out as a DC or AC measurement, as appropriate. For example a combination of AC bioimpedance measurement and DC temperature and skin moisture level measurements can be carried out in some embodiments. Making measurements for different parameters in non-overlapping time periods or in different frequency ranges (e.g., AC versus DC) avoids or minimizes interference between measurements.

In various examples, notwithstanding the appended claims, the disclosure is also defined by the following clauses:

Clause 1: A bioimpedance measurement apparatus, including a plurality of electrodes configured to be placed on respective skin portions of a subject, at least one temperature sensor probe configured to be placed on a respective skin portion of the subject and to generate a signal indicative of a temperature of the skin portion the at least one temperature sensor probe is placed on, at least one moisture sensor probe configured to be placed on a respective skin portion of the subject and to generate a signal indicative of a moisture level of the skin portion the at least one moisture sensor probe is placed on, and an electronic module configured to: determine an impedance between at least two of the plurality of electrodes, receive from the at least one temperature probe the signal indicative of the temperature of the skin portion the at least one temperature sensor probe is placed on and determine the at least one temperature of the skin portion the temperature sensor probe is placed on, receive from the at least one moisture sensor probe the signal indicative of the moisture level of the skin portion the at least one moisture sensor probe is placed on and determine the moisture level of the skin portion the at least one moisture sensor probe is placed on, and determine a bioimpedance of the subject based at least in part on the determined impedance, temperature, and moisture level.

Clause 2: The bioimpedance measurement apparatus of clause 1, wherein the at least one moisture sensor probe is adapted to be placed on substantially the same skin portion as a respectively one of the plurality of electrodes.

Clause 3: The bioimpedance measurement apparatus of clause 1 or clause 2, wherein the at least one temperature sensor probe is adapted to be placed on substantially the same skin portion as at least one of the plurality of electrodes.

Clause 4: The bioimpedance measurement apparatus of clause 2 or clause 3, further including a support, wherein the at least one moisture sensor probe and the at least one of the respective one of the plurality of electrodes are attached to the support, and the support is configured to maintain the at least one moisture sensor probe and the at least a respective one of the plurality of electrodes on the respective skin portion of the subject.

Clause 5: The bioimpedance measurement apparatus of clause 3 or clause 4, further including a support, wherein the at least one temperature sensor probe and the at least one of the respective one of the plurality of electrodes are attached to the support, and the support is configured to maintain the at least one temperature sensor probe and the at least a respective one of the plurality of electrodes on the respective skin portion of the subject.

Clause 6: The bioimpedance measurement apparatus of clause 4 or clause 5, wherein the at least one temperature sensor probe is attached to the support, and the support is further configured to maintain the at least one temperature sensor on substantially the same skin portion on which the at least one moisture sensor probe and the at least a respective one of the plurality of electrodes are maintained by the support.

Clause 7: The bioimpedance measurement apparatus of any one of clauses 1-6, wherein the electronic module is configured to determine the impedance and at least one of the temperature and moisture level at substantially the same time.

Clause 8: The bioimpedance measurement apparatus of clause 7, wherein the electronic module is configured to determine an AC impedance between at least two of the plurality of electrodes and determine the temperature from a DC signal indicative of the temperature received from the temperature sensor probe.

Clause 9: The bioimpedance measurement apparatus of clause 8, wherein the at least one temperature sensor probe includes a thermocouple.

Clause 10: The bioimpedance measurement apparatus of clause 8 or clause 9, wherein the at least one temperature sensor probe includes an infrared temperature probe.

Clause 11: The bioimpedance measurement apparatus of any one of clauses 1-10, wherein the plurality of electrodes include four electrodes, of which two current electrodes are configured to apply a current through a tissue portion between the two skin portions on which the respective current electrodes are placed on, and of which two voltage electrode are configured to be placed between the two current electrodes and sense a voltage between the voltage electrodes due to the applied current.

Clause 12: The bioimpedance measurement apparatus of any one of clauses 1-11, wherein the at least one moisture sensor probe includes a second plurality of electrodes.

Clause 13: A system for determine muscle mass of a subject, the system including: a radiographic imaging system configured to acquire a radiographic image of the subject; a bioimpedance measurement apparatus, including: a plurality of electrodes configured to be placed on respective skin portions of a subject, at least one temperature sensor probe configured to be placed on a respective skin portion of the subject and to generate a signal indicative of a temperature of the skin portion the at least one temperature sensor probe is placed on, at least one moisture sensor probe configured to be placed on a respective skin portion of the subject and to generate a signal indicative of a moisture level of the skin portion the at least one moisture sensor probe is placed on, and an electronic module configured to determine an impedance between at least two of the plurality of electrodes, receive from the at least one temperature probe the signal indicative of the temperature of the skin portion the at least one temperature sensor probe is placed on and determine the at least one temperature of the skin portion the temperature sensor probe is placed on, receive from the at least one moisture sensor probe the signal indicative of the moisture level of the skin portion the at least one moisture sensor probe is placed on and determine the moisture level of the skin portion the at least one moisture sensor probe is placed on, and determine a bioimpedance of the subject based at least in part on the determined impedance, temperature, and moisture level, and a processor configured to: receiving from the radiographic imaging system image data representing the acquired image, receiving from the electronic module the determined bioimpedance, and determining a muscle mass of the subject based at least in part on the image data and the determined bioimpedance.

Clause 14: The system of clause 13, wherein determining the muscle mass includes: determining a preliminary muscle mass based on the image data; and

adjust the preliminary muscle mass based on the determined bioimpedance.

Clause 15: The system of clause 13 or clause 14, wherein the bioimpedance measurement apparatus further includes a support, wherein the at least one moisture sensor probe and the at least one of the respective one of the plurality of electrodes are attached to the support, and the support is configured to maintain the at least one moisture sensor probe and the at least a respective one of the plurality of electrodes on the respective skin portion of the subject.

Clause 16: The system of any one of clauses 13-15, wherein the bioimpedance measurement apparatus further include a support, wherein the at least one temperature sensor probe and the at least one of the respective one of the plurality of electrodes are attached to the support, and the support is configured to maintain the at least one temperature sensor probe and the at least a respective one of the plurality of electrodes on the respective skin portion of the subject.

Clause 17: The system of clause 15 or clause 16, wherein the at least one temperature sensor probe is attached to the support, and the support is further configured to maintain the at least one temperature sensor on substantially the same skin portion on which the at least one moisture sensor probe and the at least a respective one of the plurality of electrodes are maintained by the support.

Clause 18: The system of any one of clauses 15-17, wherein the radiographic imaging system includes a dual-energy X-ray analysis (DXA) imaging system.

Clause 19: A method of bioimpedance measurement, including: measuring an impedance between two skin portions of a subject; measuring a skin temperature of the subject; measuring a skin moisture level of the subject; and determining a bioimpedance of the subject based at least in part on the measured impedance, temperature, and moisture level.

Clause 20: The method of clause 19, wherein: measuring the impedance includes placing a plurality of electrodes on respective skin portions of the subject and measure an impedance between two of the plurality of electrodes; measuring the skin temperature includes placing a temperature sensor probe on a respective skin portion in proximity of one of the plurality of electrodes and determining the skin temperature based a signal from the temperature sensor probe; measuring the skin moisture level includes placing a moisture sensor probe on a respective skin portion in proximity of the temperature sensor probe and determining the skin moisture level based a signal from the moisture sensor probe.

With at least some of the examples disclosed above, accurate bioimpedance measurements that take into account the effects of temperature and/or skin moisture content can be conveniently made. Accurate bioimpedance measurements can be used to obtain more accurate assessment of various characteristics, such as muscle mass, of subjects.

This disclosure describes some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

What is claimed is:

1. A bioimpedance measurement apparatus, comprising:
a plurality of electrodes configured to be placed on respective skin portions of a subject;
at least one temperature sensor probe configured to be placed on a respective skin portion of the subject and to generate a signal indicative of a temperature of the skin portion the at least one temperature sensor probe is placed on;
at least one moisture sensor probe configured to be placed on a respective skin portion of the subject and to generate a signal indicative of a moisture level of the skin portion the at least one moisture sensor probe is placed on; and
an electronic module configured to:
determine an impedance between at least two of the plurality of electrodes,
receive from the at least one temperature probe the signal indicative of the temperature of the skin portion the at least one temperature sensor probe is placed on and determine the at least one temperature of the skin portion the temperature sensor probe is placed on,
receive from the at least one moisture sensor probe the signal indicative of the moisture level of the skin portion the at least one moisture sensor probe is placed on and determine the moisture level of the skin portion the at least one moisture sensor probe is placed on, and
determine a bioimpedance of the subject based at least in part on the determined impedance, temperature, and moisture level.

2. The bioimpedance measurement apparatus of claim 1, wherein the at least one moisture sensor probe is adapted to be placed on substantially the same skin portion as a respectively one of the plurality of electrodes.

3. The bioimpedance measurement apparatus of claim 1, wherein the at least one temperature sensor probe is adapted to be placed on substantially the same skin portion as at least one of the plurality of electrodes.

4. The bioimpedance measurement apparatus of claim 2, further comprising a support, wherein the at least one moisture sensor probe and the at least one of the respective one of the plurality of electrodes are attached to the support, and the support is configured to maintain the at least one moisture sensor probe and the at least a respective one of the plurality of electrodes on the respective skin portion of the subject.

5. The bioimpedance measurement apparatus of claim 3, further comprising a support, wherein the at least one temperature sensor probe and the at least one of the respective one of the plurality of electrodes are attached to the support, and the support is configured to maintain the at least one temperature sensor probe and the at least a respective one of the plurality of electrodes on the respective skin portion of the subject.

6. The bioimpedance measurement apparatus of claim 4, wherein the at least one temperature sensor probe is attached to the support, and the support is further configured to maintain the at least one temperature sensor on substantially the same skin portion on which the at least one moisture sensor probe and the at least a respective one of the plurality of electrodes are maintained by the support.

7. The bioimpedance measurement apparatus of claim 1, wherein the electronic module is configured to determine the impedance and at least one of the temperature and moisture level at substantially the same time.

8. The bioimpedance measurement apparatus of claim 7, wherein the electronic module is configured to determine an AC impedance between at least two of the plurality of electrodes and determine the temperature from a DC signal indicative of the temperature received from the temperature sensor probe.

9. The bioimpedance measurement apparatus of claim 8, wherein the at least one temperature sensor probe comprises a thermocouple.

10. The bioimpedance measurement apparatus of claim 8, wherein the at least one temperature sensor probe comprises an infrared temperature probe.

11. The bioimpedance measurement apparatus of claim 1, wherein the plurality of electrodes comprise four electrodes, of which two current electrodes are configured to apply a current through a tissue portion between the two skin portions on which the respective current electrodes are placed on, and of which two voltage electrode are configured to be placed between the two current electrodes and sense a voltage between the voltage electrodes due to the applied current.

12. The bioimpedance measurement apparatus of claim 1, wherein the at least one moisture sensor probe comprises a second plurality of electrodes.

13. A system for determine muscle mass of a subject, the system comprising:
a radiographic imaging system configured to acquire a radiographic image of the subject;
a bioimpedance measurement apparatus, comprising:
a plurality of electrodes configured to be placed on respective skin portions of a subject;
at least one temperature sensor probe configured to be placed on a respective skin portion of the subject and to generate a signal indicative of a temperature of the skin portion the at least one temperature sensor probe is placed on;
at least one moisture sensor probe configured to be placed on a respective skin portion of the subject and to generate a signal indicative of a moisture level of the skin portion the at least one moisture sensor probe is placed on; and
an electronic module configured to:
determine an impedance between at least two of the plurality of electrodes,
receive from the at least one temperature probe the signal indicative of the temperature of the skin portion the at least one temperature sensor probe is placed on and determine the at least one temperature of the skin portion the temperature sensor probe is placed on,
receive from the at least one moisture sensor probe the signal indicative of the moisture level of the skin portion the at least one moisture sensor probe is placed on and determine the moisture level of the skin portion the at least one moisture sensor probe is placed on, and
determine a bioimpedance of the subject based at least in part on the determined impedance, temperature, and moisture level; and
a processor configured to:
receiving from the radiographic imaging system image data representing the acquired image;
receiving from the electronic module the determined bioimpedance; and
determining a muscle mass of the subject based at least in part on the image data and the determined bioimpedance.

14. The system of claim 13, wherein determining the muscle mass comprises:
determining a preliminary muscle mass based on the image data; and
adjust the preliminary muscle mass based on the determined bioimpedance.

15. The system of claim 13, wherein the bioimpedance measurement apparatus further comprises a support, wherein the at least one moisture sensor probe and the at least one of the respective one of the plurality of electrodes are attached to the support, and the support is configured to maintain the at least one moisture sensor probe and the at least a respective one of the plurality of electrodes on the respective skin portion of the subject.

16. The system of claim 13, wherein the bioimpedance measurement apparatus further comprise a support, wherein the at least one temperature sensor probe and the at least one of the respective one of the plurality of electrodes are attached to the support, and the support is configured to maintain the at least one temperature sensor probe and the at least a respective one of the plurality of electrodes on the respective skin portion of the subject.

17. The system of claim 15, wherein the at least one temperature sensor probe is attached to the support, and the support is further configured to maintain the at least one temperature sensor on substantially the same skin portion on which the at least one moisture sensor probe and the at least a respective one of the plurality of electrodes are maintained by the support.

18. The system of claim 15, wherein the radiographic imaging system comprises a dual-energy X-ray analysis (DXA) imaging system.

19. A method of bioimpedance measurement, comprising:
measuring an impedance between two skin portions of a subject;
measuring a skin temperature of the subject;
measuring a skin moisture level of the subject; and
determining a bioimpedance of the subject based at least in part on the measured impedance, temperature, and moisture level.

20. The method of claim 19, wherein:
measuring the impedance comprises placing a plurality of electrodes on respective skin portions of the subject and measure an impedance between two of the plurality of electrodes;
measuring the skin temperature comprises placing a temperature sensor probe on a respective skin portion in proximity of one of the plurality of electrodes and determining the skin temperature based a signal from the temperature sensor probe;
measuring the skin moisture level comprises placing a moisture sensor probe on a respective skin portion in proximity of the temperature sensor probe and determining the skin moisture level based a signal from the moisture sensor probe.
